(19) United States
(12) Patent Application Publication
(10) Pub. No.: US 2014/0288611 A1
(43) Pub. Date: Sep. 25, 2014

(54) ZERO BLOOD FLOW SENSITIVE HEART STIMULATOR

(71) Applicants: Adelina Pancheva, Sofia (BG); Vladimir Panchev, Sofia (BG); Marieta Pancheva, Sofia (BG)

(72) Inventors: Adelina Pancheva, Sofia (BG); Vladimir Panchev, Sofia (BG); Marieta Pancheva, Sofia (BG)

(21) Appl. No.: 13/261,823

(22) PCT Filed: Aug. 29, 2012
(86) PCT No.: PCT/BG2012/000023

§ 371 Date: Mar. 4, 2014

(30) Foreign Application Priority Data
Sep. 2, 2011 (BG) 111025

(51) Int. Cl. A61N 1/365

(52) U.S. Cl. CPC A61N 1/36571
USPC 607/17

(57) ABSTRACT

A zero flow responsive heart stimulator contains a zero flow sensors, which generate signals at the moment of the termination of blood inflow in the right atrium and the right ventricle, sensing this the most precisely in the places, where the sinoatrial node and atrioventricular node reside, for the right atrium and the right ventricle, respectively. The stimulator is based on our discovery, that the two nodes are the same sensors in the biological systems, based on the fact that until the filling of the said two chambers continues, the venous blood flow sucks on Bernoulli's principle the Ca and Na cations from the two nodes interstices, preventing the inward currant in their cells, thus delaying the completion of the 0-phase of their depolarization and the action potential, until the blood flow stops. Our discovery proves that the alternating flow/no flow of the blood in the orifice of superior vena cava and the entrance of the right ventricle is the real pacemaker that fires the both nodes, but not the inverse, as generally assumed, approximating the natural pacemaker to a clock mechanism. This, together with the discovered by us arteriovenous, arteriolymphatic and capillary pumps, closes the loop of the autonomous automatic functioning of the cardiovascular system, even completely denervated and in the absence of muscle contractions. It is the first devise, which activates the atrial and the ventricular contractions in a function of the zero-flow of the filling them venous blood, which is the exact imitation of the sinoatrial and atrioventricular firing.

### BACKGROUND OF THE INVENTION

1. Field of the Invention

The present invention relates to the cardiac pacemakers, which deliver to the heart electrical impulses with adjustable action interval.

2. Description of the Prior Art

The artificial pacing of the heart, where it is needed, suffers from several great fallacies in the understanding of the cardiovascular physiology, which, according to our concept, are the followings: 1. The venous return is driven by the difference between the right atrial filling pressure and the mean circulatory pressure, assisted by the venous muscle pump; 2. The local blood flow is regulated exclusively by the arterioles; 3. There is no local thermoregulation and the local blood flow regulation has nothing to do with local regulation of the temperature; 4. The heart is paced with the intrinsic rate of depolarization of the sinoatrial node (SAN), i. e. regularly timed automatic signal, modulated only by the vagal and sympathetic systems; 5. Where the pacing of the right atrium is absent the pacing is undertaken by of the atrioventricular node (AVN) or by other parts of the electrical system, which intrinsic rate is lower. This model was followed by the artificial pacemakers with adjustable rate of the impulse firing. For example U.S. Pat. No. 4,686,987, U.S. Pat. No. 4,535,774, U.S. Pat. No. 5,243,976, U.S. Pat. No 5,316,001, U.S. Pat. No. 7,231,250, and U.S. Pat. No. 7,653,437 monitor different parameters, as heart rate, cardiac output, blood flow, heart cameras dimension etc. in attempt to regulate the rate of pacing, according to the subject activity. In the U.S. Pat. No. 5,417,715 is used as parameter the right ventricle filling, by measuring its electrical impedance, and the minimum of the latter is used to determine the emitting the signal for contraction, either only of the right ventricle, or both, right atrium and ventricle, the latter with some delay. It is not clear, why the right atrium is fired to contract in the moment of ventricle's end diastole (minimum impedance), which corresponds to atrial end systole. There is no patent, in which the pacing of each of the right chambers to be subordinated only to the termination of its own filling, which is the case with their natural pacing.

The question of the venous return (point 1) is subject to three our publications: Panchev V, Suvandjieva A, and Pancheva M. The muscle pump is not an important determinant of muscle blood flow during exercise. J Appl Physiol. 99: 778, 2005; Pancheva M Panchev V, Suvandjieva A, and Levine B. Lower body negative pressure vs. lower body positive pressure to prevent cardiac atrophy after bed rest and spaceflight. What caused the controversy? J Appl Physiol 100 (3): 1090-1090, 2006; Pancheva M, Panchev V, Suvandjieva A, Krediet C, van Lieshout, and Wieli ng W. Improved orthostatic tolerance by leg crossing and muscle tensing: indisputable evidence for the arteriovenous pump existence J Appl Physiol 101(4): 1271-1272. According to our theory of the venous return, it is driven by the arterial pulsations, transmitted transmuraly to the veins on the principle of “hydraulic mutual induction” (a notion, introduced by us), similarly to the electromagnetic mutual induction in the Electrical Engineering, using the close apposition of arteries, veins and lymphatic vessels

The importance of this anatomical peculiarity for the venous return (including the lymph one) we explained for the first time. The main arteriovenous pumps reside in the extremities, where the hydraulic mutual induction is supported by the surrounding muscles, which compress arteries, veins, and lymphatic vessels one to another.

The questions of the local blood flow regulation and of the local thermoregulation (above points 2 and 3) are resolved with our theory of the local blood flow and local thermoregulation, briefly presented in Patent application WO 2010/017603 A2. As described there, using the “Stirling cycle” with carbon dioxide solution, the blood capillaries promote the blood in proportion to the positive gradient between the temperature of their surrounding tissue and that of the incoming in them blood, thus regulating the local blood flow in proportion to the thermal tissue loading. With this we proved that the local blood flow regulation is subordinated to the primary cooling function of the blood, the oxygen delivery being redundant, and occurring by diffusion through the entire arterial circuit, and not only through the capillaries (Kerger. H, Torres E I P, Rivas M, Winslow R M, Intaglieta M. Systemic and subcutaneous oxygen tension in conscious Syrian golden hamsters. Am J physiol 268(2 Pt 2): H802-10), 1995.

The above mentioned theory is based on the discovered by us anomaly of the solubility of carbon dioxide in saline around 37° C., which lead us to discover the mechanism of capillary pumping on the principle of Stirling-Malone engine (Allen P C, Paulson D N, and Wheatley J C. Some heat engine cycles in which liquids can work. Proc Natl Acad Sci USA; 78 (1): 31-35, 1981.). To that we arrived, testing our hypothesis that CO2 plays a decisive role in the thermo-baric homeostasis of all homeotherms, allowing coupling the capillary blood flow with the positive temperature gradient between their surrounding tissue and the capillary blood, which proves the primary cooling role of the latter. We measured the pressure-temperature relationship of CO2 dissolved in saline for different concentrations and plotted the first against the second. We found that in each diagram (FIG. 1 from WO 2010/017603 A2) there are several inflex points and that the first derivatives of all of them (FIG. 2) have one maximum always near 37° C., independently from the CO2 content. Thus, we discovered a physical constant, around which are concentrated the basal body temperatures of the great majority of homeotherms [closer to it for mammals (White C R and Seymour R S, Mammalian basal metabolic rate is proportional to body mass^{2/3} PNAS, vol. 100, No 7, 4046-4049, 2003 and the supportive table to it) and higher for birds, which bodies are more isolated], profiting from the increased slope in the temperature/pressure diagram at this point. For that we created the following theory: a) The capillary blood flow is promoted using the Stirling-Malone engine cycle; b) The working medium is the containing dissolved CO2 blood plasma, expanding by pressure fall, temperature elevation or by other factors reducing the CO2 solubility in it or producing additional amounts of CO2, like acids; c) The capillary glycocalyx molecular-chains (banded toward the venous limb by the erythrocyte passage) and the parachute-formed erythrocytes act as ratchet and pawl, determining their one-way motion, i. e. a capillary pump; d) The plasma expansion takes place on the account of the nanobubbles, which form at the liquid/solid interfaces between the glycocalyx molecular chains, their density increasing dramatically above 28 to 42° C. It goes on the account of the increase of the lateral size of nanobubbles, reaches a maximum at about 37° C. and then decreases at a higher temperature (Zhang X H, Li G, Wu Z H, Zhang X D, and Hu J. Effect of temperature on the morphology of nanobubbles at mica/water interface. Chinese phys. 14, 1774, 2005; Zhang, X. H.; Zhang, X. D.; Lou, S. T.; Zhang, Z. X.; Sun, J. L.; Hu, J. Electrolytically generated nanobubbles on highly orientated pyrolytic graphite surfaces. Langmuir, 20, 3813, 2004.) With this we discovered two, unsuspected until now roles of the capillary glycocalyx: 1. To be a part of the retched and pawl mechanism; 2. To create enormous solid/liquid interface, favorable for nanobubbles formation. The rapid formation and subsequent dissolution of nanobubbles is strongly catalyzed by the enzyme carbonic anhydrase.

According to our concept, the blood capillaries of all warm blooded (endothermic) animals represent the working part of “Stirling-Malone” engines with free pistons, in which the working fluid is the containing carbon dioxide blood plasma, the pistons represent the obtaining parachute-form in the capillaries red blood cells, the surrounding tissue is the external heat source, the heart is the external pump (the displacer), the skin veins and the lungs are the cool reservoir (the temperature sink), and all other veins and arteries are heat-transporting tubes and heat exchangers (regenerators) and arteriovenous pumps.

The use of CO₂—solution as working medium in a thermal engine, which we suggest that the blood capillaries are, is a great invention of the nature, remaining hidden for humans until now. This gives us the reason to claim it to be our discovery.

We suggest that the sweat glands use the same CO2—based mechanism of pressure increase by local temperature elevation, which determines their activation close to the 37° C. point through simple pressure-produced secretion. Local sweating can be produced, either by the temperature elevation of the sweat gland perfusing blood, or by local skin passive heating or deteriorated local heat dissipation (our observations). This behavior can be explained again with the discovered by us increase of the pressure slope at the 37° C. point for all concentrations. That mechanism is strongly supported by the inadequate sweat rate during hypocapnia (Robinson S M and King A B. Hypocapnia-induced increases in rectal temperature in man during heat exposure. J Appl Physiol 31: 656-658, 1971), by its increase during hypercapnia (Bullard B W. Effects of carbon dioxide inhalation on sweating. J Appl Physiol 19: 137-141, 1964), and by the local sweat gland activity due to direct effects of radiant heat (Randall W C. Local sweat gland activity due to direct effects of radiant heat. Am J Physiol 150: 365-371, 1947.). With this, we claim to have discovered the basic mechanism of sweating (for the time being believed to be reflexly activated contracting mechanism), which definitely resolves the mystery about the 37° C. set point.

Since the anomaly at the 37° C. is too small, only the human beings succeeded to profit maximally from it by creating microenvironment for their bodies, allowing them to regulate with minimal deviations from the beneficial for the brain function 37° C.—a premise for the appearance of modern humans, and our civilization.

On the base of the explained above, we suggest humans have lost their body hairiness, and have obtained the most favorable temperature for the capillary local thermoregulation and brain function, because they are the only homeotherms, which consciously maximally help their bodies (regulating their clothing) to maintain this most favorable temperature. This answers the question: why modern Humans (Homo Sapience) appeared first in Africa and than spread to Eurasia to replace the Neanderthals? According to us, it has been, because the climate allowed them in an easiest way to achieve the 37° C. set-point. We believe to be the firsts, who put an accent on this fact to be the decisive primary trigger of humans' evolutionary separation from the rest animal world.

This explains well the recent molecular genetic findings for the appearance of the human body lice some 50-100000 years ago, with its origin also in Africa (Kittler R, Kayser M, Stoneking M. Molecular evolution of Pediculus humanus and the origin of clothing. Current Biology, 13, 1414-1417, 2003.), which must coincide with the time, when humans began to wear clothing. Since this coincides with the appearance of the modern humans, the unresolved question is: what is the primary—the hairlessness or the clothing? Our theory, based on our findings, is that, due to this anomaly, on a certain evolutionary stage, Homos, living in Central Africa, where the ambient temperature is the highest and the most stable, begin to realize that when regulating the coverage of their bodies, they feel more comfortable (as our contemporaries do, not knowing in fact why), began the chain: improved dressing—37° C. setting—improved cerebral function—further sophistication of dressing—hairlessness—enablement to move to colder climates. This evolutionary chain has not been possible for the Neanderthals and other Homos, living in colder climates, which necessitated a jump in sophisticated clothing and housing to achieve the 37° C. setting and improved cerebral function, in spite that Neanderthals were with bigger brains. We postulate that the later was a result of their improved mode of life, due to the erected stand, quantitatively, on the account of the quality, due higher and more variable body temperature, specific for the animals with fur.

In the spirit of to the explained above factual state and not understanding of the mechanical auto regulation of the cardiovascular system, the entire fallacy, governing the directions of research in the domain of cardiac electrophysiology resides in the fact that SAN, as well, as AVN are regarded as being in a quiescent fluid, which mechanical state cannot influence the ionic inward currents. Thus, the researchers do without the information, which the venous inflow carries about the state of the right compartments' filling, information, from which the evolution, evidently was not want to do without.

### SUMMARY OF THE INVENTION

An object of the invention is to provide a heart stimulator, maximally analogous to the heart natural ones, the SAN and the AVN. We are the firsts to explain the expedience of the positioning of the two nodes exactly at the points where the inflowing in the right compartments venous blood has the greatest velocity and stops the last. The SAN firing is beat-to-beat subordinated to the optimal action of the right atrium, regarding its filling and the site of interaction must be the inward currents in the nodal pacemaker cells, as having the closest contact with the flowing venous blood via the nodal interstice. In the reality, the cation inward currant takes place from the nodal interstice, which is subjected to the venous blood sucking effect on their nearest myocardial surface. For SAN that is the crista terminalis-ridge, which creates turbulence, increasing the under-pressure, caused by the flowing blood on Bernoulli's principle. The said under-pressure sucks the fluid from the nodal interstice (via that of the over laying thin myocardial layer) with the contained in it Na and Ca cations, thus delaying the completion of the 0-phase of their depolarization and the action potential, until the blood flow stops. Our discovery proves that the alternating flow/no (zero) flow of the blood in superior vena cava's orifice is the real pacemaker that fires the SAN, but not the inverse, as generally assumed. The same is valid for the flow through the tricuspid valve in the entrance of the right ventricle, regarding the firing of AVN for determining the delay between the two firings. These are the sites, from where the evolution decided to take information for the both compartments' filling dynamics, as the most exact one.

In this train of thought, the rate of SAN firing is not a regularly timed automatic signal, but sequence of intervals, beat-to-beat matching the time, needed for the right atrium filling, which is venous return-dependent, and which termination in physiological conditions is marked by the inflow blood stopping. It is exactly this moment that we catch with the zero flow sensors in the entrances of the right atrium and the right ventricle, and supply the signals for this to the pulse generator means for emitting synchronous to them signals through the electrodes attached to the atria, and to the ventricles, respectively.

### BRIEF DESCRIPTION OF THE DRAWINGS

FIG. 1 is a schematic illustration of a first embodiment, a single-chamber unipolar or bipolar cardiac pacemaker, connected to a right atrium of a heart.

FIG. 2 shows the first embodiment in greater detail in a block diagram.

FIG. 3 is a schematic illustration of a second embodiment, a single-chamber unipolar or bipolar cardiac pacemaker according to the invention, connected to the right ventricle of a heart.

FIG. 4 shows the second embodiment in greater detail in a block diagram.

FIG. 5 is a schematic illustration of a third embodiment, a dual-chamber unipolar or bipolar cardiac pacemaker according to the invention, connected to the right atrium and the right ventricle of a heart.

FIG. 6 shows the third embodiment in greater detail in a block diagram.

### DESCRIPTION OF THE PREFERRED EMBODIMENTS

In FIG. 1 the cardiac pacemaker 1 is connected to a right atrium of a heart 2 with the lead 4, connected with the implanted in the crista terminalis, situated in the orifice of superior vena cava 7, zero flow sensor 3 and the lead 5, connected with the implanted in the right atrium stimulation electrode 6. Each one of the leads 4 and 5 represents two leads in the variant of a bipolar pacemaker.

In the block diagram of FIG. 2, the pacemaker 1 contains the electronics, namely: a zero flow means 8, which receives the zero flow signals from the zero flow sensor 3 via the lead 4, and after processing them, transmits the signals to the pulse generator means 9. The latter emits via the lead 5 and the electrode 6, simultaneous to the zero flow signals, electrical pulses to the right atrium. Thus, the pacemaker imitates exactly the function of the natural SAN, which is activated only by the zero-flow in the place of its situation, the rest of the adaptation of the circulatory system to the various conditions, in which it is placed, being performed automatically by the heart (including the same action of the natural AVN, if intact), the capillary pumps, the arteriovenous, and the arteriolymphatic pumps. We do not mention the nervous system, in order to demonstrate that with our suggested model the circulatory system works, even completely denervated.

In the second embodiment, shown in FIG. 3, the cardiac pacemaker 10 is connected to a right ventricle of a heart 11 with the lead 13, connected with the zero flow sensor 12, implanted in the internal myocardial wall, in the nearest possible proximity to the atrioventricular node, the coronary sinus, and the septal leaflet of the tricuspid valve, and the lead 14, connected with the stimulation electrode 15, implanted in the right ventricle. Each one of the leads 13 and 14 represents two leads in the variant of a bipolar pacemaker.

In the block diagram of FIG. 4, the pacemaker 10 contains the electronics, namely: a zero flow means 17, which receives the zero flow signals from the zero flow sensor via the lead 13, and after processing them, transmits the signals to the pulse generator means 18. The latter emits via the lead 14, simultaneous to the zero flow signals, electrical pulses to the right ventricle, to produce a systole. Thus, the pacemaker imitates exactly the function of the natural AVN, which is activated only by the zero-flow in the place of its situation, the rest of the adaptation of the circulatory system to the various conditions, in which it is placed, being performed automatically by the heart (including the same action of the natural SAN, which is, presumably, active), the capillary pumps, the arteriovenous and the arteriolymphatic pumps.

In the third embodiment, shown in FIG. 5, the cardiac pacemaker 19 is connected: First, to a right atrium of a heart 20 with the lead 22, connected with the implanted in the crista terminalis (situated in the orifice of superior vena cava 29) the zero flow sensor 21 and the lead 23, connected with the implanted in the right atrium stimulation electrode 24.; Second, to a right ventricle with the lead 26, connected with the implanted in the internal myocardial wall (in the nearest possible proximity to the atrioventricular node, the coronary sinus, and the septal leaflet of the tricuspid valve) zero flow sensor 25, and the lead 27, connected with the implanted in the right atrium stimulation electrode 28.

In the block diagram of FIG. 6, the pacemaker 19 contains the electronics, namely: First, a zero flow means 32, which receives the zero flow signals from the zero flow sensor via the lead 22, and after processing them, transmits the signals to the pulse generator means 33. The latter emits via the lead 23, simultaneous to the zero flow signals electrical pulses to the right atrium; Second, a zero flow means 30, which receives the zero flow signals from the zero flow sensor via the lead 26, and after processing them, transmits the signals to the pulse generator means 31. The latter emits via the lead 27, simultaneous to the zero flow signals, electrical pulses to the right ventricle.

Thus, the described pacemaker imitates exactly the function of both natural SAN and AVN, which are activated only by the zero-flow in the places of their situation. Such explained pacing of the heart rate through the venous return, together with the discovered by us capillary and arteriovenous pumps close the mechanical functional loop: heart—arteries—capillaries—veins—heart and insure the autonomic automatic action of the cardiovascular system, as a self supplied one, even completely denervated and in the absence of muscle contractions. The described automatism of the cardiac rhythm explains very well its acceleration, both: with the increase of venous return and with its reduction below certain limit, depending only on the time of the termination of the blood inflow. The latter occurs, either because the chamber is filled or, because the pressure in the entrance to the right atrium has become negative, before its filling, respectively.

We claim:

1. A zero flow responsive cardiac pacemaker comprising:
an electrode means for in vivo delivery of stimulation pulses to a heart;
a pulse generator means, connected to said electrode means, for emitting said stimulation pulses simultaneous with the signals supplied by the zero flow sensing means, said pulse generator means having control input;
a zero flow sensing means for generating signals identifying the zero flow of blood at a given place in the heart chambers or in superior vena cava, supplying these signals to the said control input, for generating pulses simultaneous with these zero flow signals.

2. A cardiac pacemaker, as claimed in claim 1, wherein the pulse generator means is one, with one control input.

3. A cardiac pacemaker, as claimed in claim 2, wherein the sensor of the zero flow sensing means is implanted in the grove formed by crista terminalis and sinus venosus, over the primary pacemaker cells of the sinoatrial node or at the nearest to it available place in the wall, washed by the incoming to the right atrium venous blood, said sensing means giving signals to the pulse generator means for emitting simultaneous to them signals through electrodes attached to the right, the left or the both atria.

4. A cardiac pacemaker, as claimed in claim 2, wherein the sensor of the zero flow sensing means is attached on the inner wall of superior vena cava on the nearest available place to the primary pacemaker cells of the sinoatrial node.

5. A cardiac pacemaker, as claimed in claim 3 or claim 4, wherein the sensor of the zero flow sensing means is implanted there via superior vena cava.

6. A cardiac pacemaker, as claimed in claim 2, wherein the zero flow sensing means determines the zero flow to the right atrium indirectly, by receiving signals for the minimal right atrial electric impedance or for the termination of the right atrial expansion.

7. A cardiac pacemaker, as claimed in claim 2, wherein the sensor of the zero flow sensing means is implanted in the internal myocardial wall, in the nearest possible proximity to the atrioventricular node, the coronary sinus, and the septal leaflet of the tricuspid valve or at the nearest to that available place on the right atrial septal wall, washed by the incoming to the right ventricle venous blood, said sensing means giving signals to the pulse generator means for emitting, simultaneous to the zero flow ones, signals through electrodes attached to the right, the left or the both ventricles.

8. A cardiac pacemaker, as claimed in claim 7, wherein the sensor of the zero flow sensing means is implanted there via superior vena cava.

9. A cardiac pacemaker, as claimed in claim 2, wherein the zero flow sensing means determines the zero flow in the right ventricle indirectly, by receiving signals for the minimal right ventricle electric impedance or for the termination of the right ventricle expansion.

10. A cardiac pacemaker, as claimed in claim 1, wherein the pulse generator mean are two, each having separate input and output, for emitting separate stimulation signals to the right, the left or both atria and to the right, the left or both ventricles.

11. A cardiac pacemaker, as claimed in claim 10, wherein the input of the first pulse generator is connected with a zero flow sensing means, which zero flow sensor is implanted in the grove formed by crista terminalis and sinus venosus, over the primary excitatory cells of the sinoatrial node or at the nearest to it available place on the wall washed by the incoming to the right atrium venous blood, said sensing means giving signals to the pulse generator means for emitting, simultaneous to the zero flow ones, signals through electrodes attached to the atria

12. A cardiac pacemaker, as claimed in claim 10, wherein the zero flow sensor of the zero flow sensing means is implanted in the inner wall of superior vena cava on the nearest available place to the primary pacemaker cells of the sinoatrial node or positioned there via superior vena cava.

13. A cardiac pacemaker, as claimed in claim 10, wherein the input of the second pulse generator is connected with a zero flow sensing means, which zero flow sensor is implanted in the internal myocardial wall over the atrioventricular node near the coronary sinus and the septal leaflets of the tricuspid valve, or at the nearest to that available place on the right atrial septal wall, washed by the incoming to the right ventricle venous blood, said sensing means giving signals to the pulse generator means for emitting, simultaneous to the zero flow ones, signals through electrodes attached to the right, the left or both ventricles.

14. A cardiac pacemaker, as claimed in claim 11, claim 12, or claim 13, wherein the sensor of the zero flow sensing means is implanted there via superior vena cava.

15. A cardiac pacemaker, as claimed in claim 10, wherein the zero flow sensing mean determine the zero flow to the right atrium and to the right ventricle indirectly, by receiving signals for the minimal right atrial or right ventricle electric impedance or for the termination of the right atrial or the right ventricle's expansion, respectively.
